(19) United States
(12) Patent Application Publication
(10) Pub. No.: US 2005/0228280 A1
(43) Pub. Date: Oct. 13, 2005

(54) Acquisition and display methods and systems for three-dimensional ultrasound imaging

(75) Inventors: Kutay F. Ustuner, Mountain View, CA (US); Joan Main, Mountain View, CA (US); Mani Vannan, Penn Valley, PA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/815,022

(22) Filed: Mar. 31, 2004

(51) Int. Cl. A61B 8/00

(52) U.S. Cl. 600/443; 600/447; 128/916

(57) ABSTRACT

An effective method to attain high volume rates in real-time three-dimensional ultrasound imaging is to reduce the lateral scan extent in azimuth and/or elevation. Reducing the scan extent, however, may make it difficult to determine the anatomical orientation during scan, or post-scan review. Anatomical landmark information is provided, with only a small impact on the volume rate by scanning along a two-dimensional plane with a greater lateral extent than a three-dimensional volume scan or by scanning over a three-dimensional volume with a lower resolution than a higher resolution sub-volume scan. The lower resolution three-dimensional image or the two-dimensional image scan provides anatomical landmark information. The higher resolution or smaller three-dimensional volume scan provides information for diagnosis of a specific region with three-dimensional imaging.

### BACKGROUND

The present invention relates to three-dimensional ultrasound imaging. In particular, user assistance for three-dimensional imaging is provided.

For three-dimensional imaging with an ultrasound system, a plurality of transmit and receive events are performed sequentially. Due to the speed of sound through tissue, larger volumes may take longer to scan. Changes in resolution may result in changes of scanning time for a given volume as well. However, for diagnosis, a high level of detail resolution is desired. To obtain the high level of detail resolution in a short period of time, the lateral extent of the image volume is reduced. If the volume size is reduced, the user may lose anatomical landmarks that help locate the scan region of interest. If the alternative approach of reducing resolution is used, information content is sacrificed.

### BRIEF SUMMARY

By way of introduction, the preferred embodiments described below include methods and systems for acquiring ultrasound data for display. Real time or more rapid three-dimensional imaging is provided with context information to assist the user. Anatomical landmark information is preserved by scanning along a two-dimensional plane with a greater lateral extent than a three-dimensional volume scan or by scanning over a three-dimensional volume with a lower resolution than a higher resolution sub-volume scan. The lower resolution three-dimensional image or the two-dimensional image scan provide anatomical landmark information. The higher resolution sub-volume or smaller three-dimensional volume scan provides information for diagnosis of a specific region.

In a first aspect, a method is provided for acquiring ultrasound data for display. Ultrasound energy is scanned along a two-dimensional plane over a first lateral range. Ultrasound energy is scanned over a three-dimensional volume with a second lateral range. The second lateral range of the three-dimensional volume is less than the lateral range of the two-dimensional plane.

In a second aspect, a system is provided for acquiring ultrasound data for display. A beamformer connects with the transducer. The beamformer is operable to scan along the two-dimensional plane over a first lateral range. The beamformer is also operable to interleave a scan three-dimensional volume over a second lateral range. The second lateral range is less than the first lateral range.

In a third aspect, a method is provided for acquiring ultrasound data for display. A three-dimensional volume is scanned with a first spatial resolution. A three-dimensional sub-volume within the volume is scanned with a second spatial resolution. The spatial resolution of the sub-volume scan is higher than the spatial resolution of the full volume scan.

In a fourth aspect, a system is provided for acquiring ultrasound data for display. A beamformer connects with the transducer. The beamformer is operable to scan within a three-dimensional volume with a first spatial resolution. The beamformer is also operable to interleave a scan within a three-dimensional sub-volume of the volume with a second spatial resolution. The second spatial resolution is higher than the first spatial resolution.

The present invention is defined by the following claims, and nothing in this section should be taken as limitation on those claims. Further aspects and advantages of the invention are discussed below in conjunction with the preferred embodiments and may be later claimed in combination or independently.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for acquiring ultrasound data for display;

FIG. 2 is a flowchart diagram of one embodiment of a method for acquiring ultrasound data for display;

FIGS. 3A and 3B are graphical representations of embodiments of two-dimensional scans with a greater lateral extent than associated three-dimensional scans;

FIG. 4 is a flowchart diagram of another embodiment of a method for acquiring ultrasound data for display; and

FIG. 5 is a graphical representation showing scanning a sub-volume with high resolution and scanning the rest of a volume associated with the sub-volume with a lower resolution.

### DETAILED DESCRIPTION OF THE DRAWINGS AND PRESENTLY PREFERRED EMBODIMENTS

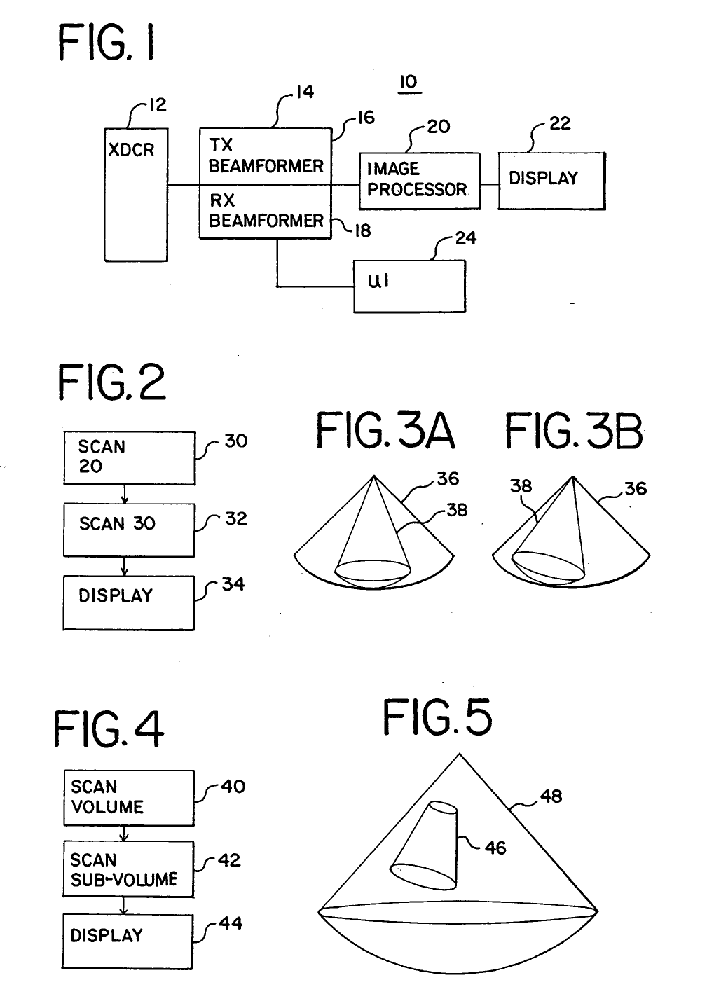

FIG. 1 shows a system 10 for acquiring ultrasound data for display. The system 10 includes a transducer 12, a beamformer 14 (e.g. a transmit beamformer 16 and/or a receive beamformer 18), an image processor 20, a display 22 and a user input 24. Additional, different or fewer components may be provided, such as including a memory for storage of ultrasound data. In one embodiment, the system 10 is a medical diagnostic ultrasound system, but the system 10 may be a workstation or other device with or without a transducer 12.

The transducer 12 is an array of piezoelectric or capacitive membrane elements. The array has a one-dimensional, two-dimensional, multi-dimensional, 1.25D, 1.5D, 1.75D, annular or other now known or later developed grid pattern of elements. In another embodiment, the transducer 12 and at least part or all of the beamformer 14 is located within a transducer assembly separable from an imaging device including the imaging processor 20. Alternatively, the transducer 12 is separable from the remainder of the system 10. In hand-held ultrasound systems, the transducer 12 may not be separable.

In one embodiment, the transducer 12 includes a position sensing device, such as a magnetic or electromagnetic coil, gyroscopes, infra-red, radiofrequency or other sensing system for determining a position relative to a patient, relative to a room or relative to another frame of reference. In alternative embodiments, the transducer 12 is free of additional position sensing devices. For example, a two-dimensional or multi-dimensional array is used to scan within three dimensions. Based on the electronic steering of the scan lines, the position of data relative to other data or scan lines is known. As another example, relative position is assumed. As yet another example, ultrasound data is processed to determine an amount and direction of translation and/or rotation. Using position sensing, a one-dimensional or other planar imaging array may be used for scanning a plurality of spatially distinct planes for three-dimensional imaging.

The beamformer 14 includes one or both of a transmit and receive beamformer 16, 18. The beamformer 14 connects with the transducer 12 for causing a transducer 12 to generate acoustic energy as well as forming the ultrasound data received in response to echo signals impinging on the transducer 12.

In one embodiment, the transmit beamformer 16 includes a waveform generator, a memory, transistors, amplifiers, delays, phase rotators, digital-to-analog converters, combinations thereof or other now known or later developed electrical signal generators for acoustic transmissions with a phased array. The transmit beamformer 16 is a beamformer disclosed in U.S. Pat. No. 5,675,554, the disclosure of which is incorporated by reference.

The receive beamformer 18 is a pre-amplifier, base band filter, filter, analog-to-digital converter, amplifier, delay, phase rotator, summer, combinations thereof or other now known or later developed electrical devices for converting phase array signals into data representing spatial locations within a scan region. In one embodiment, the receive beamformer is a receive beamformer disclosed in U.S. Pat. Nos. 5,555,534 and/or 5,685,308, the disclosures of which are incorporated herein by reference.

The beamformer 14 is operable to scan along a two-dimensional plane over a first lateral range in transmit, receive or both. For example, the beamformer 14 is operable to perform a sector scan by transmission and reception along a plurality of scan lines extending over an about a 90 degree angle. Lesser or greater angle extents may be used for a sector or Vector® image. The scan lines extend by about 45 degrees from normal along the azimuth dimension. As an alternative to varying angular ranges, the two-dimensional plane may be scanned over a lateral range defined by the azimuth extent of the transducer array for linear imaging. The beamformer 14 is also operable to scan a three-dimensional volume over another lateral range, such as steering in two dimensions or steering in one dimension with movement of the transducer 12. The lateral range of the three-dimensional volume is less than the lateral range of the two-dimensional plane along at least one dimension, such as within the two-dimensional plane. For sector or vector scans, the three-dimensional volume is associated with a lesser scan angle than the two-dimensional plane. For linear imaging, the lateral extent of the three-dimensional volume is less than of the two-dimensional plane, such as using scan lines that extend from only a portion of the azimuth length of the transducer array. The lateral extent of the scan is defined by the scan geometry, such as the placement of scan lines through focusing profiles, apodization profiles and aperture selection.

In alternative or additional embodiment, the beamformer 14 is operable to scan within a three-dimensional volume with one spatial resolution and operable to scan within a sub-volume of the three-dimensional volume with a higher spatial resolution. For example, one or more of the frequency of scanning, imaging bandwidth, aperture size, aperture location, apodization type, scan geometry and scan line density are varied depending on which portion of a three-dimensional volume is being scanned. With higher frequency, a larger aperture and a denser scan line distribution, a sub-volume is scanned with a higher spatial resolution. A lower spatial resolution volume may be more rapidly scanned than a same region with a higher spatial resolution. By scanning the sub-volume with higher spatial resolution, medical diagnosis may be improved or based on more information content. By scanning the remainder of the three-dimensional volume with a lower resolution, anatomical reference information at a lower resolution may be provided for positioning the sub-volume associated with higher resolution imaging. As a result, higher volume rate and/or real time three-dimensional imaging (e.g., four-dimensional imaging) may more likely be provided.

In either of the beamformer embodiments discussed above, the beamformer is operable to switch between parameters, such as aperture, frequency, apodization profile, delay profile and combinations thereof between transmit and receive events in order to vary a lateral extent, a scanning position, or resolution in an interleaved manner. By switching between beamforming parameters on a line by line, group of lines, full scan, multiple full scan and combinations thereof (e.g., plane scan interleaved with volume scan) basis, interleaved scanning is provided in a same imaging session and/or for presenting images at a substantially same time. Any of various combinations of scan patterns may be provided, such as scanning the entire three-dimensional volume in a low resolution mode and then transmitting additional scan lines within the sub-volume to provide a higher resolution using data from both scans. Alternatively, a three-dimensional volume is scanned in any of various patterns and when the sub-volume portion is being scanned, the beamformer parameters are switched to provide a higher resolution. In the other embodiment, the two-dimensional plane may be scanned sequentially with the smaller lateral extent three-dimensional volume. Alternatively, some of the scan line data from the two-dimensional scan or three-dimensional scan is used for forming the other of the three-dimensional or two-dimensional scans.

The image processor 20 is a detector, filter, scan converter, three-dimensional processor or other now known or later developed device for generating two-dimensional images and/or three-dimensional representations. For example, the image processor includes a B-mode detector, a Doppler detector or both B-mode and Doppler detectors. The Doppler detector detects any of variance, velocity or energy associated with the ultrasound data. The data is then scan converted on a two-dimensional basis, such as where the three-dimensional volume is scanned by a plurality of separate two-dimensional planes. Alternatively, the data is provided directly to a three-dimensional processor. The three-dimensional processor generates a three-dimensional representation using alpha blending, minimum intensity projection, maximum intensity projection, surface rendering or other now known or later developed three-dimensional rendering techniques. For the two-dimensional scan, the data representing the two-dimensional plane is processed with a scan converter, but may be processed using the three-dimensional processor to form a two-dimensional image.

The display 22 is a monitor, CRT, LCD, projector, flat screen, combinations thereof or other now known or later developed display device. The display 22 connects with the beamformer, such as through the image processor 20. The display 22 is operable to generate a two-dimensional image as a function of a two-dimensional scan and generate a three-dimensional representation as a function of a three-dimensional scan. The mapping function for the two-dimensional and the three-dimensional images may be different. For example one may use a gray scale map while the other a color map. Where the two-dimensional scan is provided with a three-dimensional scan having a lesser lateral extent, the resulting two-dimensional image and the three-dimensional representation are displayed together. For the display, the lateral extent of the two-dimensional image will be greater than the three-dimensional image. For portions of the three-dimensional representation conceptually located behind the viewed two-dimensional image, the data may be removed, ignored or included as part of the three-dimensional imaging. For example, the portions of the two-dimensional image that are not viewed through the lesser lateral extent three-dimensional volume are displayed. For all overlapping portions, the three-dimensional representation is used. For rotational viewing of the three-dimensional image, the two-dimensional image is also rotated or altered. At a certain point, the two-dimensional image may have a lesser lateral extent on the display than the three-dimensional image even though the scan to acquire the two-dimensional image had a greater lateral extent, such as where the two-dimensional image is looked at edge on. Alternatively, in real time three-dimensional imaging, the lateral axis of the two-dimensional plane for scanning the two-dimensional image is maintained orthogonal to the viewing direction. Yet in another embodiment, there may be more than one two-dimensional image each with wider lateral extents than the three-dimensional image. For example, in Cardiology application these planes may correspond to apical four-chamber and apical two-chamber views.

For displaying a high-resolution sub-volume with a lower resolution volume, two separate three-dimensional representations are formed. For any overlapping locations, the high-resolution information is used on the display. Alternatively, a single three-dimensional representation is rendered from the low resolution and high-resolution data together. For example, for alpha blending along a viewing direction, the data intersected by the viewing vector includes both low resolution as well as high-resolution data. The low and high-resolution data are blended together. The rendering algorithm may account for the differences in resolution by increasing weighting provided to the high-resolution data or other differences in processing. Alternatively, the rendering is performed without any changes as a function of high resolution or low-resolution data.

In the embodiment combining a two-dimensional image with a three-dimensional representation, a two-dimensional image is a B-mode image and the three-dimensional representation is based on Doppler data. In alternative embodiments, both of the two-dimensional image and the three-dimensional representation are the same B-mode or Doppler mode type. In yet other alternative embodiments, one or both of the two-dimensional image and three-dimensional representation are formed from the same or different one or more imaging modes, such as B-mode, tissue harmonic, Doppler velocity, Doppler energy, tissue velocity, tissue energy or other flow modes, contrast agent data, strain information, torsion information, parametric imaging, contrast pulse sequences, or other now known or later developed imaging modes.

The user input 24 is a trackball, mouse, keyboard, button, touch screen, touchpad, slider, dial or other now known or later developed user input device. The beamformer 14 is responsive to the user input 24. For example, the position of a three-dimensional scan relative to a two-dimensional scan is selected by the user, such as selecting a scan angle or other lateral extent of one or both of the two dimensional image and the three-dimensional scan. As another example, the size and the position of a sub-volume within a three-dimensional scan is selected by the user, such as selecting a lateral extent, depth or other relative positioning information. As used herein, a lateral extent corresponds to azimuth or elevation direction. The user may select a single lateral extent or may select the lateral extent along two or more dimensions. By allowing user selection of the three-dimensional imaging relative two-dimensional image or of a sub-volume relative to an entire volume, the region of particular interest associated with the three-dimensional image or the high resolution sub-volume may be accurately positioned without requiring movement of the transducer or positioning of the two-dimensional scan or low resolution volume information in an undesirable location. Alternatively, a user moves the transducer 12 without providing further user input for relative positioning.

FIG. 2 shows one embodiment of a method for acquiring ultrasound data for display. Additional, different or fewer acts may be provided in the same or different order.

In act 30, a scan is performed over a two-dimensional plane. The scan extends over a first lateral range. For example, ultrasound scan lines over a first scan angle range are provided. For sector or Vector® scans, the scan angle range is of any value, such as about 90 degrees. The 90 degrees extends 45 degrees on each side of a normal to the center of the transducer, but may extend at a greater, lesser or unequal angles relative to the transducer. The scan of the two-dimensional plane over the first lateral range is performed as a function of imaging parameters, such as the frequency, aperture, delay profile, apodization profile, scan line density or other beamformer parameters.

In act 32, a three-dimensional volume is scanned over a lateral range that is less than the lateral range of the two-dimensional scan. For example, FIGS. 3a and 3b show a two-dimensional scan 36 over about a 90-degree scan angle. The three-dimensional scan 38 is in a conical pattern with the scan angle less than 70 degrees, such as about a 45-degree scan angle. The lateral range of the two-dimensional scan 36 is greater than the lateral range of the three-dimensional scan 38 within the plane of the two-dimensional scan 36. For orthogonal to the two-dimensional plane 36, the lateral range of the three-dimensional scan is the same as the three-dimensional lateral extent within the two-dimensional plane, but may be greater than or lesser. For example, the lateral extent of the three-dimensional scan 38 orthogonal to the plane of the two-dimensional scan 36 is a lesser range than the lateral range of the three-dimensional scan 38 within the two-dimensional plane or across the two-dimensional plane.

Other beamformer parameters than the lateral range or extent of the three-dimensional scan 36 relative to the three-dimensional scan 38 may be different between the scans. For example, the scan lines or two-dimensional scan 36 is associated with a different frequency, aperture, scan geometry, scan line density, or combinations thereof than for the three-dimensional scan 38. For example, the frequency or aperture associated with the two dimensional scan is decreased, allowing a decreased scan line density and lesser spatial resolution within the plane than the three-dimensional scan also within the plane or anywhere within the three-dimensional volume. The scans of acts 30 and 32 are interleaved.

In an optional act, the lateral range of the three-dimensional scan 38, the two-dimensional scan 36 or both is set as a function of user input. For example, the user selects the size, angle, lateral range and/or the depth of the three-dimensional scan 38 by selecting a region of interest, selecting a numerical value or altering a graphic representation. Further user positioning may be provided, such as allowing the user to position the three-dimensional scan 38 relative to the two-dimensional scan 36. For example, FIG. 3b shows the three-dimensional scan 38 positioned differently relative to the two-dimensional scan 36 than shown in FIG. 3a. Alternatively, the system automatically positions the three-dimensional scan 38 at a set position or a position that adapts as a function of received data. The lateral extent and the depth may also be automatically or preset.

In act 34, a display is generated. For example, a two-dimensional image is generated as a function of the two-dimensional scan of act 30. Any of various modes of imaging may be used, such as B-mode, Doppler or other now known or later developed modes.

A three-dimensional representation is generated as a function of the three-dimensional scan of act 32. The three-dimensional representation is displayed overlapping with the two-dimensional image, but may be displayed separately. The lateral extent on the display of the two-dimensional image is greater than the three-dimensional representation due to the difference in lateral extent of scans in same displays. Where the two-dimensional image is rotated to be more edge on than orthogonal to the viewing direction, the three-dimensional image may appear to have a larger lateral extent than the two-dimensional image. In one embodiment, the three-dimensional representation is a Doppler representation, but other modes of data now known or later developed may be used for the three-dimensional representation.

Using the scans discussed above and shown in FIGS. 3A and 3B, a user may view the two-dimensional image for anatomical reference. The interleave ratio of act 30 may be reduced, such as by persisting the two-dimensional image. The viewing of the anatomical reference may allow for better positioning of the scan for the three-dimensional representation. As a result, the three-dimensional representation more likely includes diagnostically significant information. Real time or more rapid three-dimensional scanning is provided by a smaller three-dimensional volume scan without sacrificing anatomical reference information.

To more likely include anatomical reference information, a low-resolution three-dimensional scan is used instead of the two-dimensional scan. FIG. 4 shows another embodiment of a method for acquiring ultrasound data for display using a three-dimensional scan of a volume at a low resolution with a sub-volume at a high resolution. Additional, different or fewer acts may be provided in the same or different order. For example, the sub-volume scan in act 42 is performed prior to the volume scan of act 40.

In act 40, a three-dimensional volume is scanned at least in part with a first spatial resolution. For example, the aperture, frequency, scan line density, other beamformer parameter or combinations thereof are altered to provide a desired resolution, such as a low resolution. The scan is performed over a same or different lateral range over two dimensions and to a desired depth. Any of various three-dimensional volume scan geometries may be used. For example, the three-dimensional volume scan is performed over a range of scan angles, such as about 90 degrees, about 70 degrees or other scan angle ranges in both azimuth and elevation directions. The entire three-dimensional volume is scanned at a same or different low resolution than a sub-volume within the volume. Alternatively, the regions of the volume not including the sub-volume are scanned. For the sub-volume, a higher resolution is used.

In act 42, the three-dimensional sub-volume of the volume is scanned with a different or higher spatial resolution. Different higher spatial resolutions within the sub-volume may be used. The sub-volume 46 shown in FIG. 5 has a lesser lateral range along at least one or two dimensions than the volume 48. For example, the scans associated with the sub-volume 46 have a lesser range of scan angles than the scan angles associated with the scan of the volume 48. The depth or third dimension of the sub-volume scan 46 may also be different than for the volume 48. In alternative embodiments, the sub-volume 46 has a same lateral or depth extent along at least one dimension as the volume 48.

The different resolutions are provided by using a different frequency, aperture, scan geometry, scan line density or combinations thereof for scanning the sub-volume than for scanning the reminder of the volume 48. In one embodiment, the spatial resolution of the sub-volume 46 is at least one third higher than the spatial resolution for the remainder of the volume 48. In one embodiment, the entire sub-volume is scanned with a first particular resolution, and the rest of the volume 48 is scanned with a different particular spatial resolution. Alternatively, the entire volume 48 is scanned at a lower resolution, and the sub-volume 46 is scanned at the higher resolution. Line or group of line interleaving may alternatively be used.

The position of the sub-volume 46 within the volume 48 is set automatically by the system, such as being preset or set based on adaptive processes. Alternatively or additionally, the sub-volume size along one, two or three dimensions, shape, relative position or combinations thereof is set as a function of user input. For example, the user selects a region of interest in two or three dimensions and the sub-volume is positioned relative to the selected region of interest. As another example, the user selects a point and the sub-volume 46 is centered relative to that point. As shown in FIG. 5, the sub-volume is positioned off center from the volume 48 but has a lesser lateral extent along two dimensions and a lesser depth.

In act 44, a three-dimensional representation is generated based on the low spatial resolution scan of the volume. A three-dimensional representation of the sub-volume is also generated as a function of the higher spatial resolution scan. The three-dimensional representation of the sub-volume may be formed from data just using the high spatial resolution scan, or a combination of data from the scan of the volume and the high spatial resolution scan of the sub-volume. The low spatial resolution scan may occur through the sub-volume or entirely outside of the sub-volume. In either case, the three-dimensional representation generated on the screen may include data from both scans or only one scan for a given pixel or spatial location. The higher spatial resolution of the sub-volume 46 more likely includes diagnostically useful information. The lower resolution scan of the rest of the volume 48 may include diagnostic information but with lesser information content. The lesser information content allows for more rapid scans of the volume and user viewing of anatomically referenced information. The high-resolution sub-volume is positioned at the region of interest. By only scanning the sub-volume 46 with a high spatial resolution, a more rapid scan of the volume and region of interest in three dimensions may be provided.

While the invention has been described above by reference to various embodiments, it should be understood that many changes and modifications may be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for acquiring ultrasound data for display, the method comprising:
(a) scanning along a two-dimensional plane over a first lateral range with ultrasound; and
(b) scanning a three-dimensional volume over a second lateral range with ultrasound, the second lateral range less than the first lateral range within the two-dimensional plane;
wherein (a) and (b) are interleaved at least in part

2. The method of claim 1 wherein (a) comprises scanning over a first scan angle range and (b) comprises scanning over a second scan angle range.

3. The method of claim 2 wherein (a) comprises scanning over an about 90 degree sector or Vector® region.

4. The method of claim 1 wherein (b) comprises scanning the three-dimensional volume over a third lateral range perpendicular to the two-dimensional plane, the third lateral range being less than the first lateral range.

5. The method of claim 1 further comprising:
(c) generating a two-dimensional image as a function of (a); and
(d) generating a three-dimensional representation as a function of (b), a lateral extent on a display of the two-dimensional image being greater than the three-dimensional representation.

6. The method of claim 1 further comprising:
(c) generating a two-dimensional B-mode image as a function of (a); and
(d) generating a three-dimensional Doppler representation as a function of (b).

7. The method of claim 1 further comprising:
(c) setting the second lateral range as a function of user input.

8. The method of claim 1 wherein (a) comprises scanning in response to a first imaging parameter in addition to the first lateral range, and (b) comprises scanning in response to a second imaging parameter in addition to the second lateral range, the first and second imaging parameters being a same type of parameter with different values.

9. The method of claim 8 wherein (a) comprises imaging with one of: center frequency, bandwidth, aperture, apodization, scan geometry, scan line density and combinations thereof different than for (b).

10. The method of claim 1 wherein (a) comprises scanning with a higher spatial resolution than (b).

11. A system for acquiring ultrasound data for display, the system comprising:
a transducer;
a beamformer connected with the transducer, the beamformer operable to interleave a scan along a two-dimensional plane over a first lateral range and a scan of a three-dimensional volume over a second lateral range, the second lateral range less than the first lateral range within the two-dimensional plane.

12. The system of claim 11 wherein the beamformer is operable to scan over a first scan angle range as the first lateral range and scan over a second scan angle range as the second lateral range.

13. The system of claim 11 further comprising:
a display connected with the beamformer, the display operable to generate a two-dimensional image as a function of the scan over the first lateral range and to generate a three-dimensional representation as a function of the scan over the second lateral range, a lateral extent on a display of the two-dimensional image being greater than the three-dimensional representation.

14. The system of claim 13 wherein the two-dimensional image comprises a B-mode image and the three-dimensional representation comprises a Color Doppler image.

15. The system of claim 11 further comprising:
a user input, the second lateral range being a function of data from the user input.

16. The system of claim 11 wherein the beamformer is operable to scan an outer region of the two-dimensional plane with a higher resolution than the scan of the three-dimensional volume.

17. The system of claim 11 further comprising a user input, the steering angle of the three-dimensional volume being a function of data from the user input.

18. A method for acquiring ultrasound data for display, the method comprising:
(a) scanning within a three-dimensional volume with a first spatial resolution; and
(b) scanning within a three-dimensional sub-volume of the volume with a second spatial resolution, the second spatial resolution higher than the first spatial resolution;
wherein (a) and (b) are acquired within a same imaging session.

19. The method of claim 18 wherein (b) comprises scanning within the entire three-dimensional sub-volume at the second spatial resolution and (a) comprises scanning at the first spatial resolution within the entire three-dimensional volume other than the sub-volume.

20. The method of claim 18 wherein (a) comprises scanning over a first lateral range and (b) comprises scanning over a second lateral range, the second lateral range less than the first lateral range along at least one dimension.

21. The method of claim 20 wherein the sub-volume has lesser lateral range along three dimensions than the volume.

22. The method of claim 20 wherein (a) comprises scanning over a first range of scan angles and (b) comprises scanning over a second range of scan angles, the second range less than the first range.

23. The method of claim 18 further comprising:
(c) generating a first three-dimensional representation as a function of (a); and
(d) generating a second three-dimensional representation as a function of (b), the second three-dimensional having a higher spatial resolution than the first three-dimensional representation.

24. The method of claim 18 further comprising:
(c) setting the sub-volume size as a function of user input.

25. The method of claim 18 wherein (a) comprises scanning with one of: center frequency, bandwidth, aperture, apodization, scan geometry, scan line density and combinations thereof different than for (b).

26. The method of claim 18 wherein the second spatial resolution is greater than ⅓ the first spatial resolution along at least one dimension.

27. A system for acquiring ultrasound data for display, the system comprising:
a transducer;
a beamformer connected with the transducer, the beamformer operable to interleave a scan within a three-dimensional volume with a first spatial resolution and a scan within a three-dimensional sub-volume of the volume with a second spatial resolution, the second spatial resolution higher than the first spatial resolution.

28. The system of claim 27 further comprising:
a user input, the beamformer responsive to data from the user input indicating one of a size and position of the sub-volume relative to the volume.

29. The system of claim 27 wherein the beamformer is operable to scan at the first and second spatial resolutions in response to different values for at least one of: center frequency, bandwidth, aperture, apodization, scan geometry and scan line density.
